(19) United States
(12) Patent Application Publication
(10) Pub. No.: US 2005/0010371 A1
(43) Pub. Date: Jan. 13, 2005

(54) Workflow-based research system with genetic pedigree display and organism tracking

(76) Inventor: Christopher Scott Merriam-Leith, Nampa, ID (US)

Correspondence Address: CHRISTOPHER SCOTT MERRIAM-LEITH, 7450 Northern Dancer Court, Nampa, ID, 83686, US

(21) Appl. No.: 10/882,314

(22) Filed: Jul. 2, 2004

Related U.S. Application Data
(60) Provisional application No. 60/481,073, filed on Jul. 9, 2003.

(51) Int. Cl. G06F 19/00; G01N 33/48; G01N 33/50

(52) U.S. Cl. 702/20

(57) ABSTRACT

A computer system for facilitating genetic research includes a project workflow module to define plural workflow steps; a workflow control module to provide the workflow steps and capturing research data; and a data store to store the data. The system can further include a genetic pedigree display module to display, in a genealogical orientation, entity nodes, and for at least one of the nodes, a trait symbol representing at least one trait possessed by the respective node. A trait symbol can be a trait tail, and the display module can employ parametric geometry. The system can also include an organism identification module to access an organism identifier corresponding to an organism, and to associate a representation of the identifier with a medium. The representation can be a barcode symbol or value.

### Cross-Reference to Related Documents

This document claims the priority benefit, and incorporates by reference in its entirety, U.S. Provisional Patent Application No. 60/481,073, entitled Transgenic Model Management System, and filed on Jul. 9, 2003 by CHRISTOPHER SCOTT MERRIAM-LEITH.

### FIELD OF THE INVENTION

The present invention relates to research systems, and more particularly, to research systems that capture data and visually convey genetic information.

### BACKGROUND OF THE INVENTION

The development of genetic research technologies and the creation and utilization of genetically modified organisms have both come to play an important role in today's biomedical research. Over the last decade, transgenic organisms have been one of the most useful resources for creating laboratory models in the study of human disease. Utilization of transgenic technology in research over the last decade clearly demonstrates the dependence of medical research on this new field of science. In more recent years, therapeutic development has changed with the introduction of new Knockout techniques. These new techniques now permit the creation of transgenic organisms with the deletions of specific genes. As a result of these innovations, both the importance and amount of data arising from genetic research has increased substantially.

Additionally, the current environment has inherently spawned significant limitations in regards to managing transgenic models at various stages of development. Researchers have no ability to track, sort, or analyze the attributes related to the vectors within the various steps of transgenic development. Managing existing projects without initially performing a cumbersome analysis of the various data sources has proven most problematic, in terms of managing large amounts of research data and realizing the synergistic value of data compilations. Further, the current environment lacks the ability to trigger alerts, or to provide audit ability for the tracking of target data. Also, there is a limited consistency of data attributes and types available for concrete analyses.

Moreover, there lacks an ability to analyze or sort attributes existing external to present data environments, and no provision for continuity across plural research areas. The overall project flow can be impeded, as a practical and fundamental matter, as a result of limitations relating to locating all attributes to any specific phenotypic analysis of any particular transgenic model.

To further compound these research difficulties, FDA clinical data requirements and the importance of regulatory compliance with 21 CFR Part 11, combined with the need for accurate information has increased the critical demand for an integrated system with efficient data access. Overlaying all of this is the demanding nature of today's competitive marketplace and the urgency to accelerate discovery and development in order to bring effective new therapies to market faster.

Therefore, it would desirable to provide a workflow-based research system. It would also be desirable to provide such a system having a genetic pedigree display. It would be even further desirable to provide such a system having organism tracking.

### BRIEF SUMMARY OF THE INVENTION

It is, therefore, an object of the invention to provide a workflow-based research system.

It is another object of the invention to provide a workflow-based research system with a genetic pedigree display.

It is another object of the invention to provide a workflow-based research system with organism tracking.

These and other objects of the present invention are accomplished by the workflow-based research system and method disclosed herein.

The present invention can be embodied in a computer system and a method for facilitating genetic research.

In an exemplary embodiment of the invention, a computer for facilitating genetic research can include the following: a project workflow module that defines a plurality of workflow steps for developing a genetically modified organism; a workflow control module adapted to provide, via a graphical user interface, the plurality of workflow steps to a researcher to allow the researcher to input research data; and a data store adapted to store the research data. The following are exemplary aspects of the invention: the project workflow module can be based, for example, on one of a transgenic model, a knockout model, and a breeding model; and the plurality of workflow steps can be predefined or user-configurable.

Additionally, and independently, in another exemplary embodiment, the system can further include a genetic pedigree display module adapted to display, in a genealogical orientation, a plurality of entity nodes, and for at least one of said plurality of nodes, to display, in orientation with the respective node, a trait symbol representing at least one trait possessed by the respective node; where the trait symbol represents each of the at least one trait with a respective graphical representation, the plurality of entity nodes includes at least one of a male sex node, a female sex node, and an unknown sex node, and the displayed nodes and trait symbols collectively provide a genetic pedigree.

The following are also exemplary aspects of the invention: the genetic pedigree can be, for example, a genogram; the graphical representation can include a unique color code, for example; the trait symbol can represent a plurality of traits possessed by the respective node; the trait symbol can be a trait tail; the genetic pedigree display module can employ parametric geometry; and genetic pedigree display module can be further adapted to display, for at least one of said plurality of nodes, in orientation with the respective node, a trait tail representing one or more traits possessed by the respective node.

Alternatively, in yet another exemplary embodiment of the invention, the system can further include an organism identification module adapted to access an organism identifier corresponding to a selected genetically modified organism, and to associate a representation of the identifier with a medium. The following are exemplary aspects of the invention: the representation can be at least one of a barcode symbol and a barcode value; the medium can be a card adapted to be physically associated with a cage; and the medium can be one of a first selected type and a second selected type.

In a further exemplary embodiment of the invention, in a genetic research facilitating computer system, a method can include the following acts: defining a plurality of workflow steps for developing a genetically modified organism; providing, via a graphical user interface, said plurality of workflow steps to a researcher to allow the researcher to input research data; and storing the research data in a data store. The following are exemplary aspects: the plurality of workflow steps can be based on one of a transgenic model, a knockout model, and a breeding model; and the plurality of workflow steps can be one of predefined and user-configurable.

Additionally, and independently, in still yet a further exemplary embodiment of the invention, the method can further include displaying a genetic pedigree, including displaying, in a genealogical orientation, a plurality of entity nodes; and for at least one of said plurality of nodes, displaying, in orientation with the respective node, a trait symbol representing at least one trait possessed by the respective node; where the trait symbol represents each of the at least one trait with a respective graphical representation, and the plurality of entity nodes includes at least one of a male sex node, a female sex node, and an unknown sex node. The following are exemplary aspects of the invention: the genetic pedigree can be a genogram; the graphical representation can include a unique color code; the trait symbol can represent a plurality of traits possessed by the respective node; the trait symbol can be a trait tail; the genetic pedigree is displayed through use of parametric geometry; and displaying the genetic pedigree can further include for at least one of said plurality of nodes, displaying, in orientation with the respective node, a trait tail representing one or more traits possessed by the respective node.

Alternatively, in still yet another exemplary aspect of the invention, the method can further include providing an identification medium, including accessing an organism identifier corresponding to a selected genetically modified organism; and associating a representation of said organism identifier with the identification medium. The following are exemplary aspects of the invention: the representation can be one of a barcode symbol and a barcode value; the identification medium can be a card adapted to be physically associated with a cage; and the identification medium can be one of a first selected type and a second selected type.

### BRIEF DESCRIPTION OF THE DRAWINGS

The present invention is illustrated by way of example, and not limitation, in the figures of the accompanying drawings, in which:

FIG. 1 illustrates an exemplary computer system having a project workflow module, a workflow control module, and a data store.

FIG. 2 illustrates an exemplary project workflow module being based on a transgenic, knockout, or breeding model.

FIG. 3 illustrates another exemplary computer system additionally having a genetic pedigree display module and/or an organism identification module.

FIG. 4 illustrates an exemplary genetic pedigree provided by a genetic pedigree display module, and having a plurality of entity nodes and exemplary trait symbols.

FIG. 5 illustrates an exemplary legend identifying exemplary aspects of a genetic pedigree.

FIG. 6 illustrates an exemplary screen shot of an exemplary aspect of the present invention, in which an organism identification module provides a representation of an organism identifier in association with a medium.

FIG. 7 is a flow diagram illustrating workflow capture of the present invention.

FIG. 8 is a flow diagram illustrating workflow capture in combination with the displaying of a genetic pedigree having a plurality of entity nodes and at least one trait symbol.

FIG. 9 is a flow diagram illustrating workflow capture in combination with the displaying of a genetic pedigree having a plurality of entity nodes, at least one trait symbol, and at least one trait tail.

FIG. 10 is a flow diagram illustrating workflow capture in combination with the providing of an identification medium.

### DETAILED DESCRIPTION OF THE INVENTION

The invention will now be described in more detail by way of example with reference to the embodiments shown in the accompanying figures. It should be kept in mind that the following described embodiments are only presented by way of example and should not be construed as limiting the inventive concept to any particular physical configuration or order.

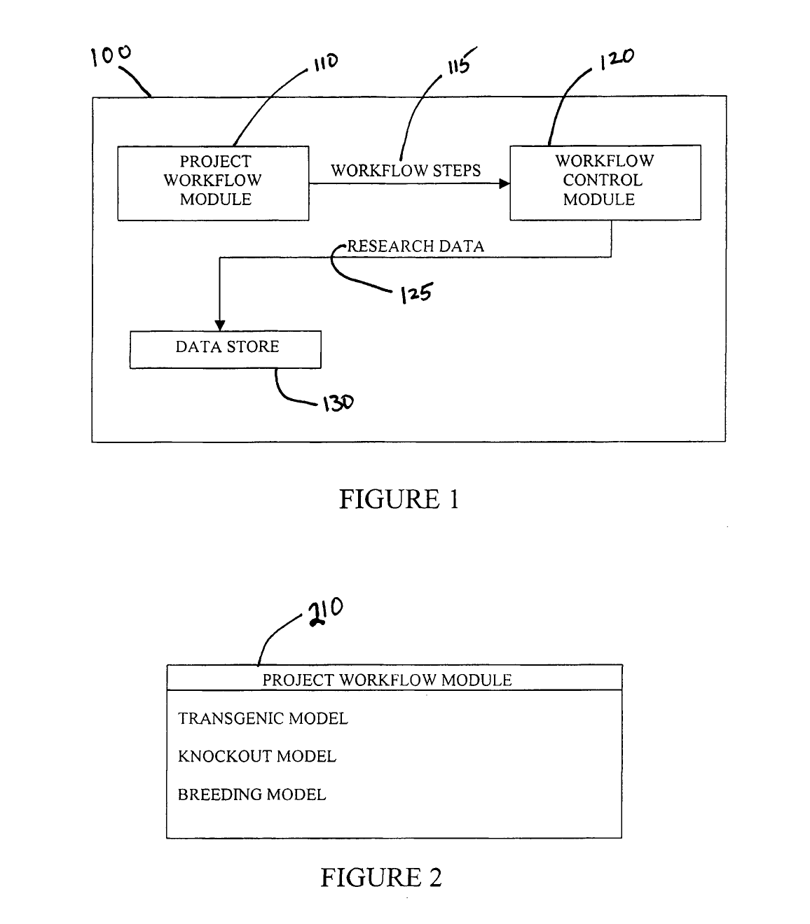

FIG. 1 illustrates an exemplary embodiment of the present invention, in which a computer system 100 for facilitating genetic research includes a project workflow module 110 that defines a plurality of workflow steps 115 for developing a genetically modified organism; a workflow control module 120 that is adapted to provide, via a graphical user interface (not shown), the plurality of workflow steps to a researcher to allow the researcher to input research data 125; and a data store 130 adapted to store the research data.

As shown in FIG. 2, project workflow module 110 can be based on a particular model. For example, as shown in FIG. 2, a model can include a transgenic model, knockout model, or a breeding model, for example and not in limitation. Accordingly, workflow steps 115, which can be predefined or user-configurable, will be based on the particular model.

Thus, the present invention can support genetic research workflow types beyond those specifically enumerated herein, including variations thereof.

In an exemplary embodiment of the present invention, a transgenic-based workflow can include procedural steps for creating a transgenic model using a pronuclear injection methodology. Exemplary steps are as follows:
1) Define Project
2) Make Transgenic Injection Request
3) Perform Superovulation
4) Perform Injection and input into log
5) Perform Animal Transfer and Tracking
6) Perform Transgene Analysis
7) Record Transgene Analysis Results

In another exemplary embodiment of the present invention, a knockout-based workflow can include procedural steps for creating a knockout model using an ES Cell and Blastocyst injection methodology. Exemplary steps are as follows:
1) Define Project
2) Make ES Cell Request
3) Make Blastocyst Injection Request
4) Perform Superovulation
5) Perform Injection and input into log
6) Perform Animal Transfer and Tracking
7) Perform Transgene Analysis
8) Record Transgene Analysis Results

In yet another exemplary embodiment of the present invention, a breeding-based workflow can include procedural steps for propagating the development of both transgenic and knockout models using transgene positive founders. Exemplary steps are as follows:
1) Define Project
2) Make Breeding Request
3) Setup Cage Management
4) Perform Animal Transfer and Tracking
5) Perform Transgene Analysis
6) Record Transgene Analysis Results
7) Perform Rederivation
8) Perform Cryopreservation
9) Make Specimen Request

It should be noted that steps 7-9 are not uniquely related to a breeding workflow, but can be performed at any point during the performance of any workflow methodology.

Referring again to FIG. 1, in still yet another exemplary aspect of the invention, workflow control module 120 provides workflow steps 115, via a graphical user interface (not shown) to a researcher (not shown), such that the researcher can input research data 125 during a research session. Data store 130 is adapted to store research data 125, and can be, for example and not in limitation, a relational database

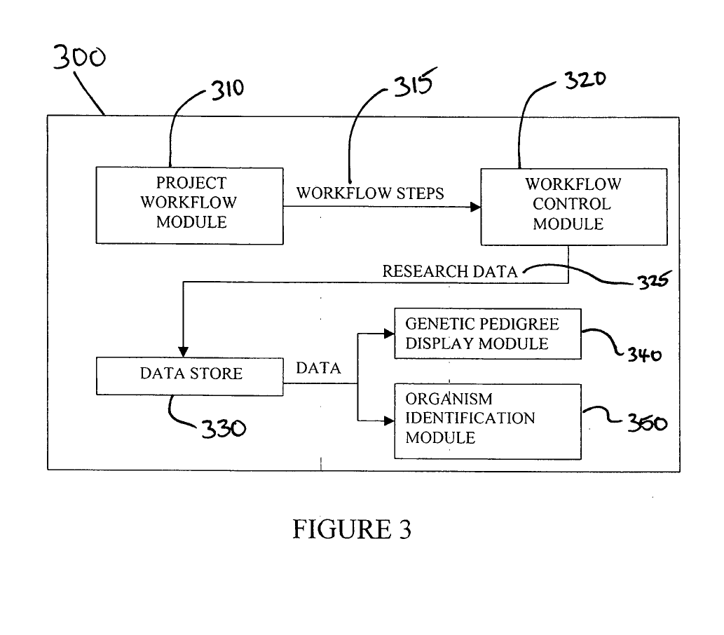

As shown in FIG. 3, system 100 can further include a genetic pedigree display module 340 and an organism identification module 350.

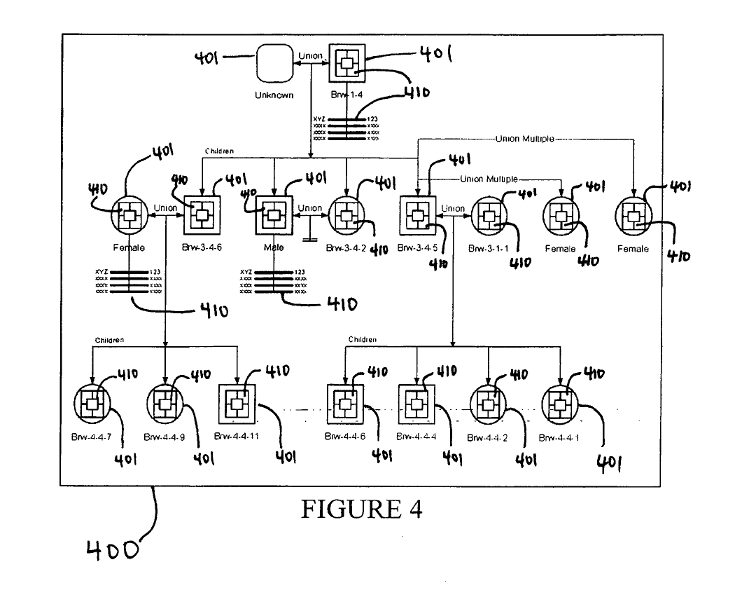

Referring to FIGS. 3 and 4, genetic pedigree display module 340 is adapted to display a genetic pedigree 400, which includes a plurality of entity nodes 401 in a genealogical orientation. As further shown, genetic pedigree module 340 is also adapted to display, for at least one of the plurality, a trait symbol 410 in orientation with the respective node. Trait symbol 410, such as the examples shown, represent at least one trait possessed by the respective node in orientation therewith. As further shown, the plurality of entity nodes 401 include at least one of a male sex node, a female sex node, and an unknown sex node. Through trait illustration and propagation of an exemplary embodiment of the present invention, enhanced information can be provided to provide a highly advantageous resource for genetic research.

In one exemplary embodiment, genetic pedigree 400 can be a genogram.

In another exemplary embodiment, genetic pedigree display module 340 can employ parametric geometry. In still yet another exemplary embodiment, genetic pedigree display module 340 can employ the VISIO software product produced by MICROSOFT CORPORATION.

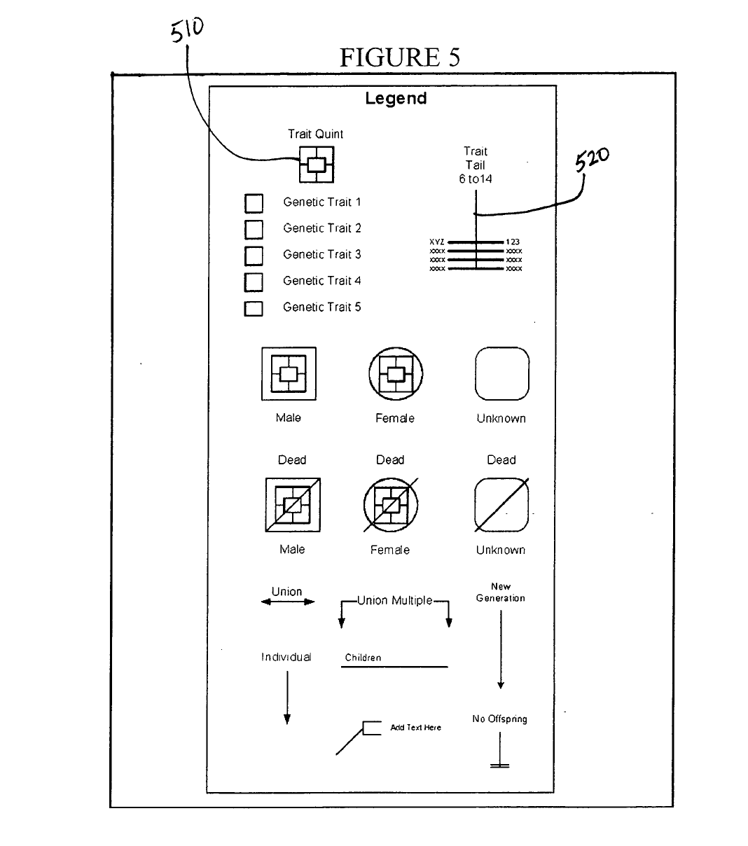

Reference is now made to FIG. 5, which illustrates an exemplary legend for optional use with genetic pedigree 400. As shown in FIGS. 4 and 5, trait symbol 410 can be a trait quint 510 or a trait tail 520, FIG. 5 does not show these references for example and not in limitation. Further, trait quint 510, for example, can visually represent up to five traits, while trait tail 520 can represent up to eight traits. Further, particular traits represented by trait quint 510 or trait tail 520 can include a unique color code, such that particular traits can be visually tracked more easily through genetic pedigree 400. It should be noted, however, that while particular ranges have been illustratively provided, trait symbol 520 is not so limited, and in particular, can represent at least one trait. Further, a union multiple can allow an organized way to associate multiple unions to an individual represented in a genetic pedigree.

In another exemplary embodiment, genetic pedigree display module 340 can independently be the basis for a system and method in and of itself.

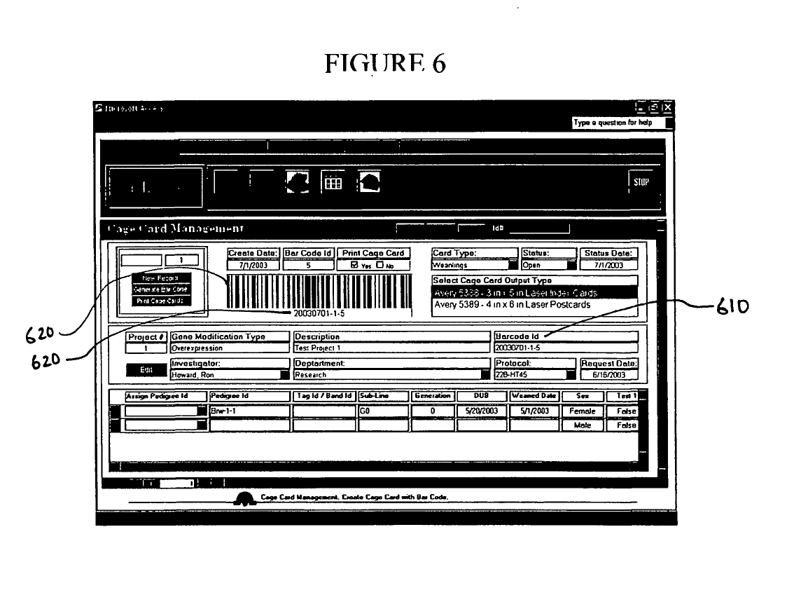

Referring to FIGS. 3 and 6, according to another exemplary embodiment, computer system 300 can further include an organism identification module 350, which is adapted to access an organism identifier 610 and to associate a representation (not shown) thereof with a medium (not shown). As illustrated in FIG. 6, organism identifier 610 is a value associated with a particular organism, and can be a Barcode ID 610, for example and not in limitation. A representation 620 of organism identifier 610 can be, for example and not in limitation, a bar code number 620 and/or a bar code value 620. Association can be through any means consistent with the spirit of the invention, such as via printing. A medium (not shown) can be, for example and not in limitation, a card; and further, can be physically associated with a particular organism's cage, such that the organism's identification can be readily determined by using the representation to reference the identification.

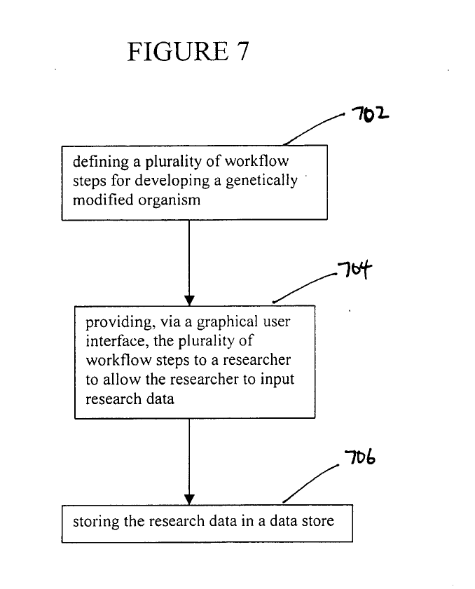

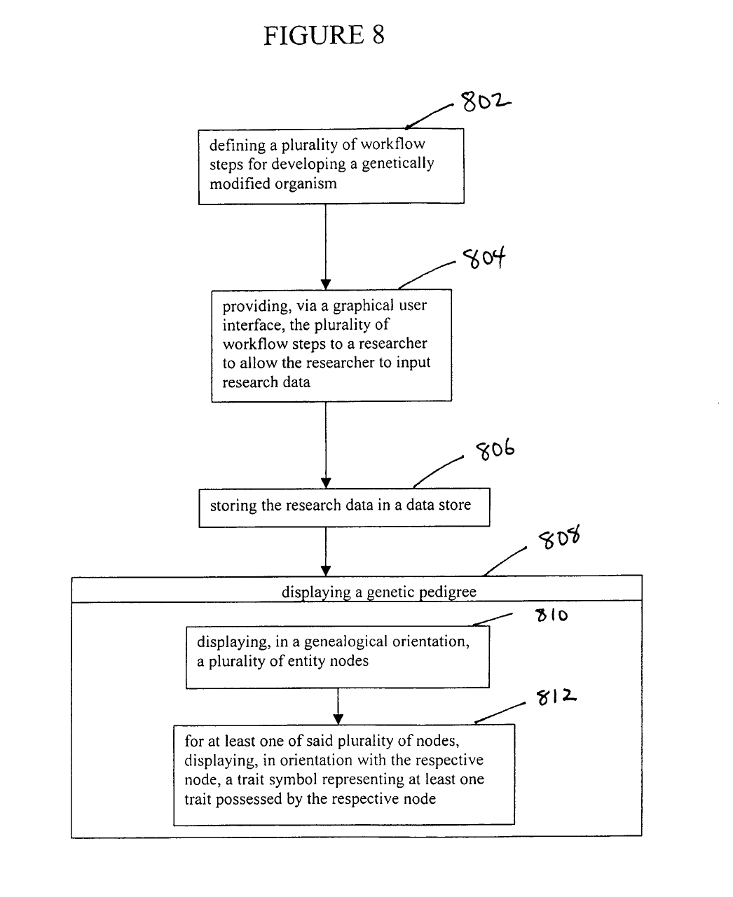

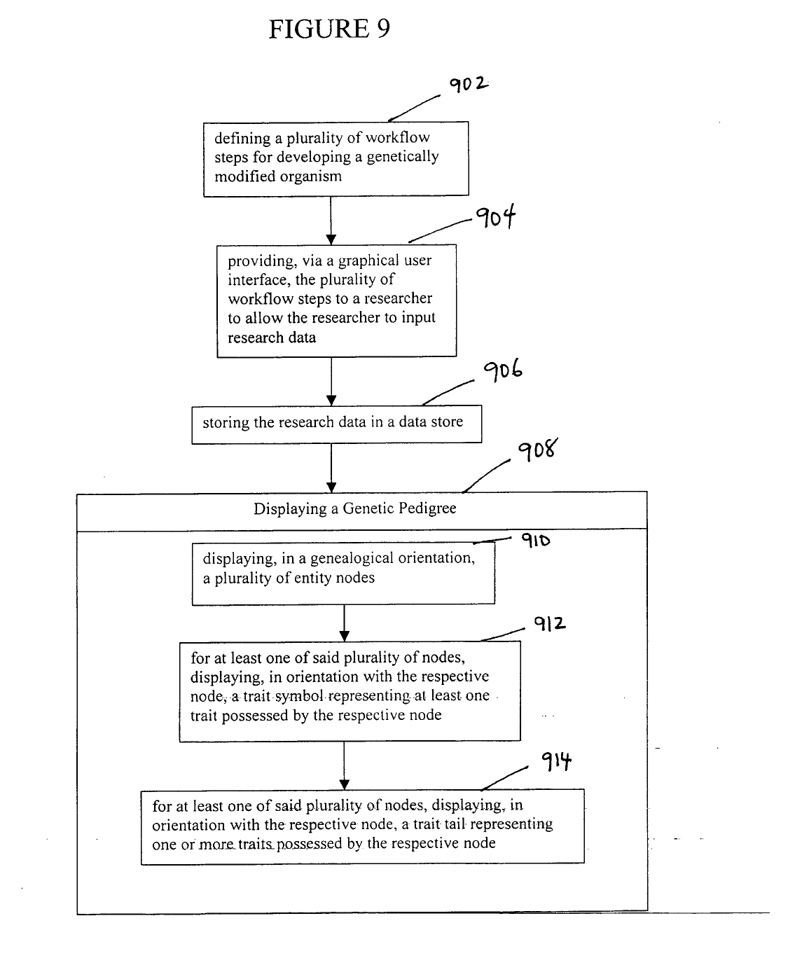

FIG. 7 illustrates another exemplary embodiment of the invention, in which, a method in a genetic research facilitating computer system can include the following acts: defining a plurality of workflow steps for developing a genetically modified organism (block 702); providing, via a graphical user interface, the plurality of workflow steps to a researcher to allow the researcher to input research data (block 704); and storing the research data in a data store (block 706). As illustrated in FIG. 8, the method can further include displaying a genetic pedigree (block 808), which includes displaying, in a genealogical orientation, a plurality of entity nodes (block 810); and for at least one of said plurality of nodes, displaying, in orientation with the respective node, a trait symbol representing at least one trait possessed by the respective node (block 812). As illustrated in FIG. 9, displaying a genetic pedigree (block 908) can further include for at least one of said plurality of nodes, displaying, in orientation with the respective node, a trait tail representing one or more traits possessed by the respective node (block 914).

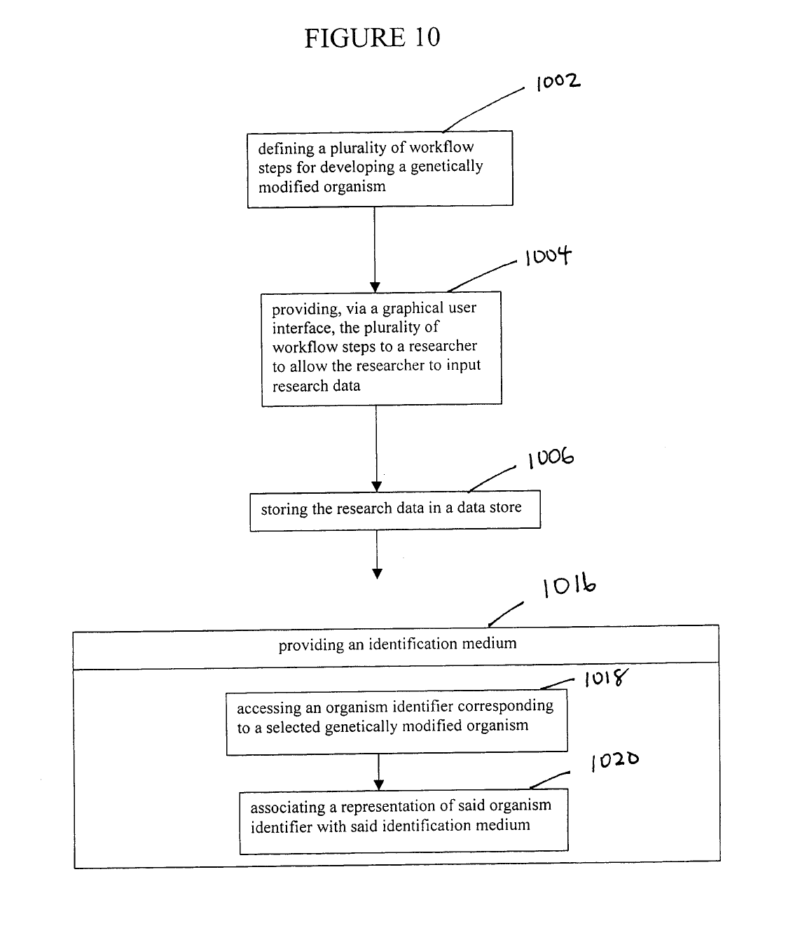

Alternatively, in still yet another exemplary aspect of the invention illustrated in FIG. 10, the method can further include providing an identification medium (block 1016), including accessing an organism identifier corresponding to a selected genetically modified organism (1018); and associating a representation of said organism identifier with said identification medium (1020).

According to still yet a further exemplary embodiment, the present invention can provide the capability to perform linkage analysis and the generation of pedigree charts and genograms. The present invention can utilize VISIO, for example and not in limitation, to create advanced pedigree charts. The integration of VISIO can provide an innovative way to graphically display and/or view the genetic history of genetic models. In this exemplary embodiment, genetic pedigree display module 340 can combine programmable shapes developed with a template to create drawings in which the shapes are used. Further, genetic pedigree display module 340 can utilize automation to control the shapes, drawings, properties, and attributes of entity nodes 401. For example and not in limitation, genetic pedigree display module 340 can be implemented with VISUAL BASIC FOR APPLICATIONS by MICROSOFT CORPORATION. In still yet a further exemplary embodiment, genetic pedigree display module 340 can include a combination of shapes and an automation program that creates a genetic history using a genogram chart, and can include a stencil of master shapes (“masters”) that a user can drag and drop onto the drawing page to create a drawing—without having to draw anything manually. Genetic pedigree display module 340 can also use automation to dynamically create the genogram charts, which provides a significant innovative breakthrough in this field of science by, for example, giving a user the instant ability to chart genetic pedigrees for analysis purposes. Genetic pedigree display module 340 can also include a template that provides a stencil of specific shapes for use in new drawings, and that predefines the drawing scale, drawing size, grid size, paper size for printing, styles, and layers. In still a further exemplary aspect, a stencil can include the following symbols (see FIG. 5):
1. Trait Quint
2. Trait Tail
3. Male
4. Female
5. Unknown
6. Dead Male
7. Dead Female
8. Dead Unknown
9. Union
10. Union Multiple
11. New Generation
12. Individual
13. Children
14. Text Call Out
15. No Offspring

In still yet a further exemplary aspect of the invention, genetic pedigree display module 340 can offer a user access to sophisticated graphics functionality with its drawing tools. Further, shapes can be programmed by means of formulas within a ShapeSheet window, for example and not in limitation. One or more master shape can include an assortment of formulas that represents its attributes, such as its width, height, and behavior, such as what the shape does when a user double-clicks it, for example and not in limitation. In still yet another exemplary aspect of the invention, genetic pedigree display module 340 master shapes can be parametric. That is, a master shape can adjust its geometry and other attributes according to parameters defined by genetic pedigree display module 340. Thus, rather than fixed geometry based on hard-coded x-y coordinates, masters shape geometry can be based on formulas that recalculate dynamically. Thus, in this exemplary embodiment, genetic pedigree display module 340 master shapes can encapsulate behavior on a drawing page. In another exemplary aspect, a master shape can be construed as a component whose default behavior is provided by genetic pedigree display module 340, and whose particular behavior is defined by the values contained within data store 130. Further, a master shape can have its own ShapeSheet spreadsheet, which defines the shape's particular shape, behavior, and/or capabilities. Thus, a ShapeSheet spreadsheet can be construed as a property sheet of a shape, in which each property is set by,a value or formula that is recalculated dynamically as the user works with the shape. Via a ShapeSheet window, viewing and editing a shape's formulas and/or features can be achieved. For example and not in limitation, via a ShapeSheet window and automation, the following can be controlled:
1) Geometry, flipping, rotation, visible or hidden paths
2) Color, pattern, and line weight
3) Text, including font, paragraph formatting, and orientation
4) Control handles that help users adjust the shape
5) Connection points where other shapes can be glued
6) Custom properties that can contain user data

It will be apparent to one skilled in the art that the manner of making and using the claimed invention has been adequately disclosed in the above-written description of the exemplary embodiments and aspects taken together with the drawings.

It should be understood, however, that the invention is not necessarily limited to the specific embodiments, aspects, arrangement, and components shown and described above, but may be susceptible to numerous variations within the scope of the invention. For example, although the above-described exemplary aspects of the invention are believed to be particularly well-suited for facilitating genetic research involving animals and other non-human organisms, the present invention can equally facilitate genetic research involving humans. Accordingly, the specification and drawings are to be regarded as illustrative and enabling, rather than restrictive.

Therefore, it will be understood that the above description of the embodiments of the present invention are susceptible to various modifications, changes, and adaptations, and the same are intended to be comprehended within the meaning and range of equivalents of the appended claims.

1. A computer system for facilitating genetic research, comprising:
a project workflow module that defines a plurality of workflow steps for developing a genetically modified organism;
a workflow control module adapted to provide, via a graphical user interface, the plurality of workflow steps to a researcher to allow the researcher to input research data; and
a data store adapted to store the research data.

2. The system of claim 1, wherein said project workflow module is based on one of a transgenic model, a knockout model, and a breeding model.

3. The system of claim 1, wherein the plurality of workflow steps are one of predefined and user-configurable.

4. The system of claim 1, further comprising
a genetic pedigree display module adapted to display, in a genealogical orientation, a plurality of entity nodes, and for at least one of said plurality of nodes, to display, in orientation with the respective node, a trait symbol representing at least one trait possessed by the respective node;
wherein the trait symbol represents each of the at least one trait with a respective graphical representation, the plurality of entity nodes includes at least one of a male sex node, a female sex node, and an unknown sex node, and the displayed nodes and trait symbols collectively provide a genetic pedigree.

5. The system of claim 4, wherein the genetic pedigree is a genogram.

6. The system of claim 4, wherein the graphical representation includes a unique color code.

7. The system of claim 4, wherein the trait symbol represents a plurality of traits possessed by the respective node.

8. The system of claim 4, wherein the trait symbol is a trait tail.

9. The system of claim 4, wherein said genetic pedigree display module is further adapted to display, for at least one of said plurality of nodes, in orientation with the respective node, a trait tail representing one or more traits possessed by the respective node.

10. The system of claim 4, wherein said genetic pedigree display module employs parametric geometry.

11. The system of claim 1, further comprising:
an organism identification module adapted to access an organism identifier corresponding to a selected genetically modified organism, and to associate a representation of the identifier with a medium.

12. The system of claim 11, wherein the representation is at least one of a barcode symbol and a barcode value.

13. The system of claim 11, wherein the medium is a card adapted to be physically associated with a cage.

14. The system of claim 11, wherein the medium is one of a first selected type and a second selected type.

15. In a genetic research facilitating computer system, a method, comprising:
defining a plurality of workflow steps for developing a genetically modified organism;
providing, via a graphical user interface, said plurality of workflow steps to a researcher to allow the researcher to input research data; and
storing the research data in a data store.

16. The method of claim 15, wherein said plurality of workflow steps are based on one of a transgenic model, a knockout model, and a breeding model.

17. The method of claim 15, wherein said plurality of workflow steps are one of predefined and user-configurable.

18. The method of claim 15, further comprising
displaying a genetic pedigree, including
displaying, in a genealogical orientation, a plurality of entity nodes; and
for at least one of said plurality of nodes, displaying, in orientation with the respective node, a trait symbol representing at least one trait possessed by the respective node;
wherein said trait symbol represents each of the at least one trait with a respective graphical representation, and said plurality of entity nodes includes at least one of a male sex node, a female sex node, and an unknown sex node.

19. The method of claim 18, wherein the genetic pedigree is a genogram.

20. The method of claim 18, wherein the graphical representation includes a unique color code.

21. The method of claim 18, wherein said trait symbol represents a plurality of traits possessed by the respective node.

22. The method of claim 18, wherein said trait symbol is a trait tail.

23. The method of claim 18, wherein displaying the genetic pedigree further includes
for at least one of said plurality of nodes, displaying, in orientation with the respective node, a trait tail representing one or more traits possessed by the respective node.

24. The method of claim 18, wherein the genetic pedigree is displayed through use of parametric geometry.

25. The method of claim 15, further comprising:
providing an identification medium, including
accessing an organism identifier corresponding to a selected genetically modified organism; and
associating a representation of said organism identifier with said identification medium.

26. The method of claim 25, wherein said representation is one of a barcode symbol and a barcode value.

27. The method of claim 25, wherein said identification medium is a card adapted to be physically associated with a cage.

28. The method of claim 25, wherein said identification medium is one of a first selected type and a second selected type.

29. A computer system for facilitating genetic research, comprising:
a genetic pedigree display module adapted to display, in a genealogical orientation, a plurality of entity nodes, and for at least one of said plurality of nodes, to display, in orientation with the respective node, a trait symbol representing at least one trait possessed by the respective node;
wherein the trait symbol represents each of the at least one trait with a respective graphical representation, the plurality of entity nodes includes at least one of a male sex node, a female sex node, and an unknown sex node, and the displayed nodes and trait symbols collectively provide a genetic pedigree.

30. The system of claim 29, wherein the genetic pedigree is a genogram.

31. The system of claim 29, wherein the graphical representation includes a unique color code.

32. The system of claim 29, wherein the trait symbol represents a plurality of traits possessed by the respective node.

33. The system of claim 29, wherein the trait symbol is a trait tail.

34. The system of claim 29, wherein said genetic pedigree display module is further adapted to display, for at least one of said plurality of nodes, in orientation with the respective node, a trait tail representing one or more traits possessed by the respective node.

35. The system of claim 29, wherein said genetic pedigree display module employs parametric geometry.

36. In a genetic research facilitating computer system, a method of graphically displaying a genetic pedigree, comprising:
displaying a plurality of entity nodes in a genealogical orientation;
for at least one of said plurality of nodes, displaying, in orientation with the respective node, a trait symbol representing at least one trait possessed by the respective node;
wherein said trait symbol represents each of the at least one trait with a respective graphical representation, and said plurality of entity nodes includes at least one of a male sex node, a female sex node, and an unknown sex node.

37. The method of claim 36, wherein the genetic pedigree is a genogram.

38. The method of claim 36, wherein the graphical representation includes a unique color code.

39. The method of claim 36, wherein said trait symbol represents a plurality of traits possessed by the respective node.

40. The method of claim 36, wherein said trait symbol is a trait tail.

41. The method of claim 36, further comprising
for at least one of said plurality of nodes, displaying a trait tail representing one or more traits possessed by the respective node.

42. The method of claim 36, wherein said genetic pedigree is displayed through use of parametric geometry.
